(19) United States
(12) Patent Application Publication
(10) Pub. No.: US 2012/0010409 A1
(43) Pub. Date: Jan. 12, 2012

(54) IMPROVED CHEMICAL SYNTHESIS OF DIAZAINDOLES BY CHICHIBABIN CYCLIZATION

(75) Inventors: George E. Lee, Bridgewater, NJ (US); Frederick L. Shrimp, II, Bridgewater, NJ (US); Franz J. Weiberth, Bridgewater, NJ (US)

(73) Assignee: SANOFI, Paris (FR)

(21) Appl. No.: 13/257,476

(22) PCT Filed: Mar. 18, 2010
(86) PCT No.: PCT/US10/27755

§ 371 Date: Sep. 19, 2011

Related U.S. Application Data
(60) Provisional application No. 61/161,440, filed on Mar. 19, 2009.

(30) Foreign Application Priority Data
Dec. 15, 2009 (FR) 0958975

(51) Int. Cl. C07D 487/04

(52) U.S. Cl. 544/350

(57) ABSTRACT

An improved synthesis method for making diazaindoles using a Chichibabin cyclization is disclosed. In particular, this method is useful for making the compound of Formula I.

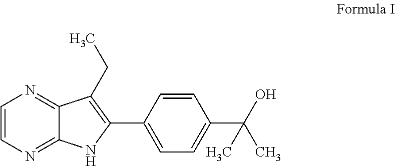

### FIELD OF THE INVENTION

This invention is directed to an improved method of synthesis for making diazaindoles.

### BACKGROUND OF THE INVENTION

The compound of general formula (I):

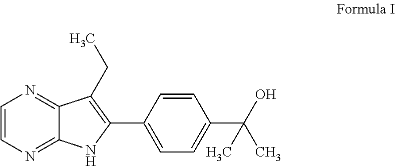

which also may be known as: 2-[4-(7-Ethyl-5H-pyrrolo[2,3-b]pyrazin-6-yl)-phenyl]-propan-2-ol and having Chemical Abstracts Service Registry Number 1011732-96-9 is known from International Application WO 2008/033798 to be an inhibitor of the Syk kinase (spleen tyrosine kinase). The synthesis of this compound is known to a person skilled in the art from the WO 2008/033798 publication.

The synthesis of this and similar compounds can be improved by the use of the synthetic scheme described here.

This and other advantages of the present invention will become apparent from the detailed description provided herein.

### SUMMARY OF THE INVENTION

The present invention provides an improved method for synthesizing diazaindoles. The improved method is summarized in the reactions of Scheme 1 below.

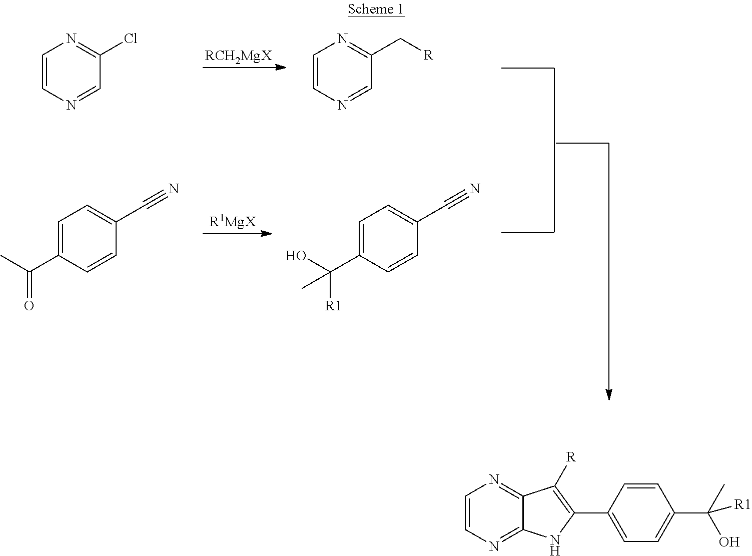

A new synthesis of the compound of formula I, shown in Scheme 2 below, has been developed and is disclosed here. In this route, n-propylpyrazine is prepared via a cross-coupling reaction of 2-chloropyrazine with n-PrMgCl. The carbinol intermediate is prepared by treating 4-acetylbenzonitrile with MeMgX. These two intermediates, in the presence of base, undergo a Chichibabin cyclization to give the compound of formula I directly. KHMDS (potassium hexamethyl disilazide) is the preferred base and TBME (tert-butyl methyl ether) is the preferred solvent for the cyclization. Several features of this new synthesis represent significant improvements compared to the original route reported in the WO 2008/033798 publication. These include: a fraction distillation is eliminated during isolation of n-propylpyrazine, an improved purity profile is obtained for the final step, the new synthesis is more convergent, and the overall yield is improved (40% vs. 15%).

The improved synthesis may be used with a variety of Grignard reagents known in the art, and with a variety of substitutions at R and R¹ in the general reaction scheme. For example, the synthetic scheme 1 is contemplated wherein: R=alkyl; R¹=alkyl, aryl or heteroaryl.

### DETAILED DESCRIPTION OF THE INVENTION

Thus, in one aspect, the present invention is directed to an improved synthesis of a compound of general formula (I):

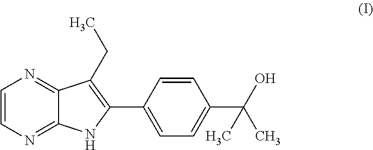

which also may be known as: 2-[4-(7-Ethyl-5H-pyrrolo[2,3-b]pyrazin-6-yl)-phenyl]-propan-2-ol.

In the present specification, the term “compound of formula I”, and equivalent expressions, are meant to embrace a compound of general formula I as hereinbefore described, which expression includes the prodrugs, the pharmaceutically acceptable salts, and the solvates, e.g. hydrates, where the context so permits. Similarly, reference to intermediates, whether or not they themselves are claimed, is meant to embrace their salts, and solvates, where the context so permits. For the sake of clarity, particular instances when the context so permits are sometimes indicated in the text, but these instances are purely illustrative and it is not intended to exclude other instances when the context so permits.

In another aspect of the invention, an improved synthesis of diazaindoles in general is provided using the general reaction scheme 1.

### DEFINITIONS

As used above, and throughout the description of the invention, the following terms, unless otherwise indicated, shall be understood to have the following meanings:—

“Pharmaceutically acceptable ester” refers to esters that hydrolyze in vivo and include those that break down readily in the human body to leave the parent compound or a salt thereof, Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Exemplary esters include formates, acetates, propionates, butyrates, acrylates, ethylsuccinates, and the like.

“Pharmaceutically acceptable prodrugs” as used herein refers to those prodrugs of the compounds of the present invention which are, within the scope of sound medical judgment, suitable for use in contact with the tissues of patients with undue toxicity, irritation, allergic response, and the like, commensurate with a reasonable benefit/risk ratio, and effective for their intended use of the compounds of the invention. The term “prodrug” refers to compounds that are rapidly transformed in vivo to yield the parent compound of the above formula, for example by hydrolysis in blood. Functional groups that may be rapidly transformed, by metabolic cleavage, in vivo form a class of groups reactive with the carboxyl group of the compounds of this invention. They include, but are not limited to such groups as alkanoyl (such as acetyl, propanoyl, butanoyl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds of this invention are cleaved in vivo, the compounds bearing such groups act as pro-drugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion is provided in Design of Prodrugs, H. Bundgaard, ed., Elsevier (1985); Methods in Enzymology; K. Widder et al, Ed., Academic Press, 42, 309-396 (1985); A Textbook of Drug Design and Development, Krogsgaard-Larsen and H. Bandaged, ed., Chapter 5; “Design and Applications of Prodrugs” 113-191 (1991); Advanced Drug Delivery Reviews, H. Bundgard, 8 , 1-38, (1992); J. Pharm. Sci., 77.,285 (1988); Chem. Pharm. Bull., N. Nakeya et aI, 32, 692 (1984); Pro-drugs as Novel Delivery Systems, T. Higuchi and V. Stella, 14 A.C.S. Symposium Series, and Bioreversible Carriers in Drug Design, E. B. Roche, ed., American Pharmaceutical Association and Pergamon Press, 1987, which are incorporated herein by reference.

“Pharmaceutically acceptable salts” refers to the relatively non-toxic, inorganic and organic acid addition salts, and base addition salts, of compounds of the present invention. These: salts can be prepared in situ during the final isolation and purification of the compounds. In particular, acid addition salts can be prepared by separately reacting the purified compound in its free base form with a suitable organic or inorganic acid and isolating the salt thus formed. Exemplary acid addition salts include the hydrobromide, hydrochloride, sulfate, bisulfate, phosphate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactiobionate, sulfamates, malonates, salicylates, propionates, methylene-bis-B-hydroxynaphthoates, gentisates, isethionates, di-p-toluoyltartrates, methanesulfonates, ethanesulfonates, benzenesulfonates, p-toluenesulfonates, cyclohexylsulfamates and laurylsulfonate salts, and the like. See, for example S. M. Berge, et al., “Pharmaceutical Salts,”J. Pharm. Sci., 66 1-19 (1977). Base addition salts can also be prepared by separately reacting the purified compound in its acid form with a suitable organic or inorganic base and isolating the salt thus formed. Base addition salts include pharmaceutically acceptable metal and amine salts. Suitable metal salts include the sodium, potassium, calcium, barium, zinc, magnesium, and aluminum salts. The sodium and potassium salts are preferred. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which have sufficient basicity to form a stable salt, and preferably include those amines which are frequently used in medicinal chemistry because of their low toxicity and acceptability for medical use. ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine, and dicyclohexylamine, and the like.

### EMBODIMENTS

With reference to inventions described herein, below are particular embodiments related thereto.

In particular, the present invention is made clear by the following example, while not being limited to the specific final product of this example.

### EXAMPLE 1

### Synthesis of the Compound of Formula I, 2-[4-(7-Ethyl-5H-pyrrolo[2,3-b]pyrazin-6-yl)-phenyl]-propan-2-ol

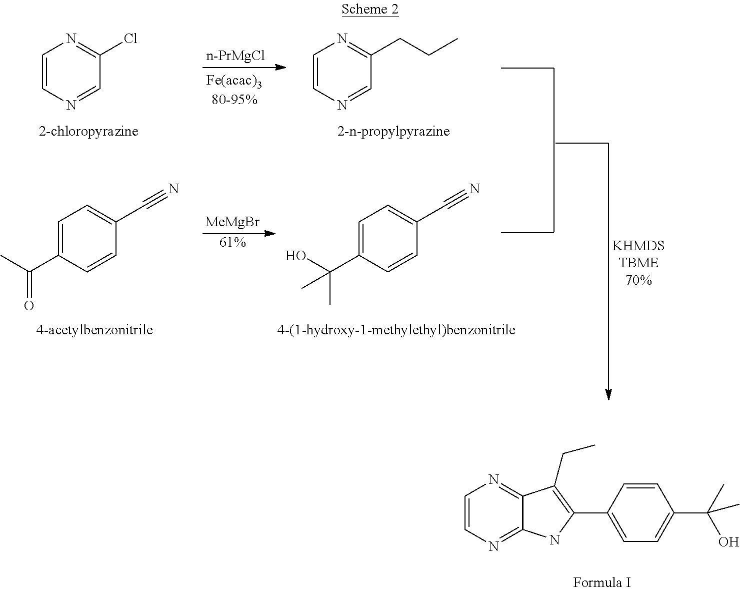

The title compound was synthesized by the reactions of Scheme 2, shown above.

2-Propylpyrazine was first prepared by the following. This was accomplished by adding to a stirred round bottom flask 8.0 mL of 2-chloropyrazine, 1.58 g Fe(acac)₃ (also known as iron acetylacetonate) and 100 mL THF (tetrahydrofuran). This was stirred under nitrogen giving a red solution. The flask was cooled in an ice-water bath for ten minutes. Then the addition of 49 mL of n-propylmagnesium chloride to the flask was begun. This resulted in a dark purple solution. After 1.5 hours 10 mL of n-propylmagnesium chloride were added over ten minutes. After an additional 20 minutes, an additional 5 mL of n-propylmagnesium chloride were added. After about 30 minutes of stirring, 22 mL of saturated aqueous NH₄Cl were added over 7 minutes. After an additional 7 mL of the NH₄Cl were added, the stirring was stopped and the mixture was allowed to stand at room temperature overnight under nitrogen.

After adding 125 mL EtOAc and 450 mL water, the contents of the flask were filtered through polypropylene and poured into a separatory funnel. The phases were separated, and the aqueous phase was extracted twice more with 125 mL portions of EtOAc. The combined organic phase was filtered through Celite® and subsequently concentrated by rotary evaporation (200 mbar, 40° C.). After a short-path distillation, the distillate was distilled (200 mbar, 90-110° C.) through a Vigreux column to give 9.0 g (82.4% yield) of 2-n-propylpyrazine.

HPLC of the final product gave a retention time of 3.0 minutes using an Elipse XDB-C8 column, 4.6×150 mm, 5 microns, using 60:40 acetonitrile/water, with 1% TFA, isocratically at 1 mL/minute at 35° C.

### Synthesis of 4-(1-Hydroxy-1-methyl-ethyl-benzonitrile

A 2000 mL round bottom flask with septum was charged with 4-acetylbenzonitrile (150g, 1032 mmole) and TBME (900 mL).

To a 5L reaction flask under nitrogen charge, TBME (2100 mL) and 3M methyl magnesium chloride in Et₂O (378 mL, 1136 mmole, 1.1 eq) were added via syringe and cooled to 17° C. The 4-acetylbenzonitrile solution was added via cannula, accompanied by an exotherm, with immediate formation of a solid slurry. Additional Grignard (36 ml, 0.1 eq) and THF 500 ml were then added with an exotherm to 23° C. At this point 500 mL saturated aqueous NH₄Cl and 1000 mL H₂O were added. The TBME phase was separated. The product was obtained through short path distillation head at 1-2 mbar/130-135° C. with an oil bath 165-180° C.

An 88% yield resulted. The 4-(1-Hydroxy-1-methyl-ethyl-benzonitrile product had a retention time of 1.9 minutes by HPLC as described above.

### Synthesis of 2-[4-(7-Ethyl-5H-pyrrolo[2,3-b]pyrazin-6-yl)phenyl]propan-2-ol

A round bottom flask was charged with 2-propylpyrazine (106 g, 868 mmole, 2 eq) and TBME (1400 mL) and solid 4-(1-hydroxy-1-methylethyl)benzonitrile (70 g, 434 mmole) was added to give a colorless solution. To the flask was then added solid KHMDS (346 g, 1738 mmole, 4 eq) over 5 minutes to give a purple slurry. This produced an exotherm to 26° C., becoming a thin slurry, readily stirred. After stirring for 48 hours, the reaction mixture was diluted with 400 mL water, slurried at 20° C., filtered and the filter cake was washed with water and TBME. The light yellow product was suction dried over night. The 2-[4-(7-Ethyl-5H-pyrrolo[2,3-b]pyrazin-6-yl)phenyl]propan-2-ol product had a retention time of 1.52 minutes by HPLC as described above. MS: 282.13 (MH⁺). Yield was 71%.

The present invention may be embodied in other specific forms without departing from the spirit or essential attributes thereof.

What is claimed is:

1. A method of making an azaindole, comprising:
a) contacting 2-chloropyrazine with a Grignard reagent to produce an alkyl pyrazine: 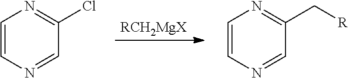
b) contacting 4-acetylbenzonitrile with a Grignard reagent to produce a carbinol: 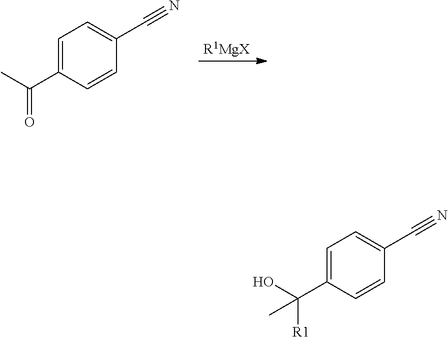
c) contacting the alkyl pyrazine with the carbinol to produce a diazaindole: 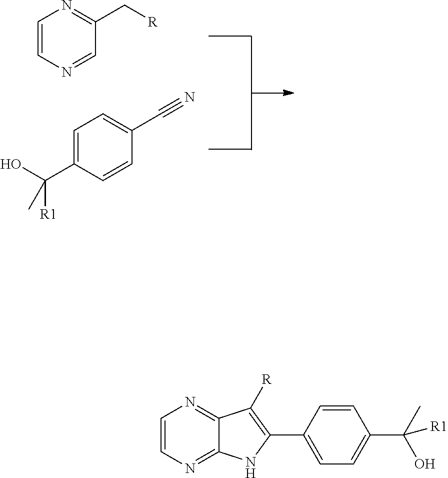
wherein:
R=alkyl;
R¹=alkyl, aryl or heteroaryl.

2. The method of claim 1, wherein the reactions are:

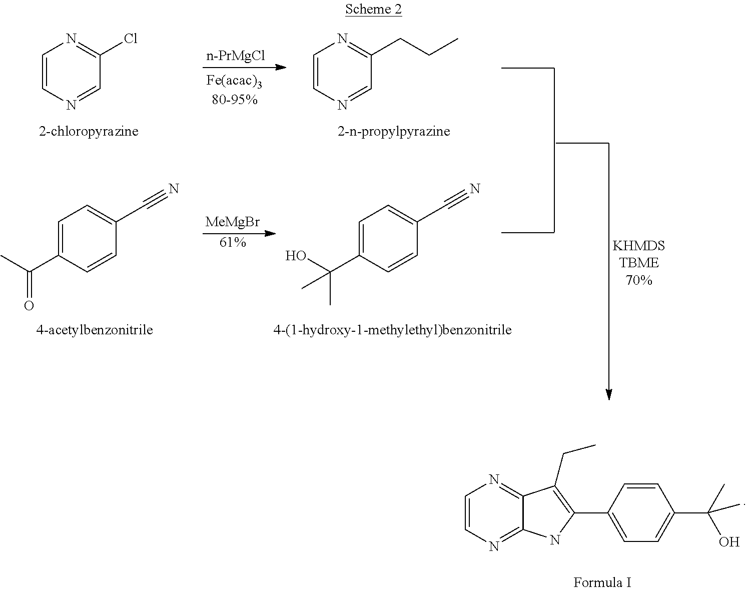

3. The method of claim 1, wherein the azaindole is made by contacting 2-n-propylpyrazine with 4-(1-hydroxy-1-methylethyl)benzonitrile.
